(19) United States
(12) Patent Application Publication
(10) Pub. No.: US 2011/0111064 A1
(43) Pub. Date: May 12, 2011

(54) Anti-Influenza Virus Agent, Anti-RS Virus Agent, and Anti-Immunodeficiency Virus Agent

(75) Inventor: Kuniaki Nerome, Okinawa (JP)

(73) Assignee: THE INSTITUTE OF BIOLOGICAL, Okinawa (JP)

(21) Appl. No.: 13/003,353

(22) PCT Filed: Jul. 8, 2009
(86) PCT No.: PCT/JP2009/062405

§ 371 Date: Jan. 10, 2011

(30) Foreign Application Priority Data
Jul. 9, 2008 (JP) 2008-179095

(51) Int. Cl. A61K 36/58; A61K 36/185; A61P 31/16

(52) U.S. Cl. 424/725

(57) ABSTRACT

The present invention herein provides an anti-influenza virus agent, an anti-RS virus agent or an anti-immunodeficiency virus agent, which comprises components originated from plants and which has excellent infectivity-inhibitory effect and proliferation-inhibitory effect against influenza viruses, RS viruses and immunodeficiency viruses. The anti-influenza virus agent, anti-RS virus agent or anti-immunodeficiency virus agent of the present invention comprises an extract derived from, or dry powder of the leaves of a plant belonging to the family Betulaceae and/or a plant belonging to the family Meliaceae.

### TECHNICAL FIELD

The present invention relates to an anti-influenza virus agent suitable for use as a disinfectant for influenza virus, or a prophylactic or a remedy for influenza virus-related diseases and in particular, to an anti-influenza virus agent, which comprises a component originated from a plant and which has an excellent viral infection-inhibitory action and a viral proliferation-inhibitory action. The present invention also relates to an anti-RS virus agent suitable for use as a disinfectant for RS virus, or a prophylactic or a remedy for RS virus-related diseases and, in particular, to an anti-RS virus agent, which comprises a component originated from a plant and which has an excellent viral infection-inhibitory action and a viral proliferation-inhibitory action. Moreover, the present invention further relates to an anti-immunodeficiency virus agent suitable for use as a disinfectant for immunodeficiency virus, or a prophylactic or a remedy for immunodeficiency virus-related diseases and, in particular, to an anti-immunodeficiency virus agent, which comprises a component originated from a plant and which has an excellent viral infection-inhibitory action and a viral proliferation-inhibitory action.

### BACKGROUND ART

The influenza is the most widely spreading virus-infectious disease and it has still been prevailing all over the world even in the present century. However, the inoculation with a vaccine prior to the outbreak of the infection for the prevention of the same is one of the central pillars of the basic preventive measures against the influenza virus-infectious diseases. The strategy second to the foregoing is the use of a therapeutic agent typified by Tamiflu (registered trade mark) and Relenza (registered trade mark). However, the prevailing virus highly frequently undergoes mutational changes and therefore, in most of cases, the effects of vaccines are greatly affected by such mutational changes. Moreover, the aforementioned therapeutic agents are in general used, in the clinical practice, at an instance when the distinct or sufficient symptom has appeared in a patient after a predetermined time period has passed since the infection with influenza virus and therefore, such a strategy is not satisfied at all. Furthermore, these therapeutic agents are relatively expensive and accordingly, it is not realistic from the economical standpoint to use the same as a disinfectant against the avian influenza virus, which has become a world-wise problem to be solved. Up to now, there has not yet been discovered or developed any prophylactic and/or therapeutic agent against influenza virus-related diseases or a disinfectant for the influenza virus, which can be used instead of the foregoing two strategies against the influenza virus-infectious diseases. If there are effectively usable prophylactics and/or therapeutic agents or disinfectants and any measure for the worldwide usage thereof is established, the population of the infected persons irrespective of the inside and outside of the country can be reduced to about a third and the rate of death from the influenza virus infection can likewise be reduced to a significantly low level.

The RS virus (respiratory syncytial virus: RSV) has been known as a principal causative virus for infantile acute respiratory infectious diseases (such as bronchiolitis and pneumonia). The RS virus has a single-stranded minus (−) RNA as a gene and is a virus belonging to the family Paramyxoviridae. The RS virus attacks the respiratory tract through physical contact and/or the droplet infection, causes such symptoms as fever, snivel, cough after the elapse of the incubation period for several days and these symptoms in general disappear within 1 to 2 weeks. In case of the infants of not older than 2-year-old and the aged, however, the virus often attacks even the lower respiratory tract through the affection of the upper respiratory tract and results in the crisis of bronchiolitis and pneumonia. In particular, in case of the infants of not older than 6-month-old, the symptoms of such diseases may be severer to such an extent that they require the hospital treatment.

There has not yet been established any effective method for treating the RS virus infectious disease in the serious condition. In this connection, a therapeutic method comprising administering a Ribavirin-containing aerosol through the spray thereof has been applied, for trial, but it has not yet been decided that such a therapeutic method shows any significant effect. In addition, the mechanism of anti-viral action of Ribavirin has not yet been elucidated and this drug suffers from a problem such that the use thereof is accompanied by a quite serious side effect such as the occurrence of anemia or oligochromemia.

On the other hand, AIDS (acquired immune deficiency syndrome) can be listed as another incurable disease with which we have now been confronted. The crisis of AIDS is observed when human immunocytes are infected with immune-deficiency virus (HIV) belonging to Retroviruses and the infected immunocytes are broken.

The population of HIV-affected persons has increased in all the world including Japan and the population of the patients suffering from AIDS has correspondingly increased. However, the prophylactic and/or therapeutic methods for AIDS are limited to ones which comprise the administration of, for instance, drugs obtained through chemical synthetic methods. In addition, the causative virus is extremely liable to undergo mutational changes and therefore, it would be quite difficult to develop any vaccine or any therapeutic agent. For this reason, there has eagerly been desired for the development of effective techniques for treating and preventing AIDS through the use of an anti-immunodeficiency virus agent.

The drugs which have already been put on the market include, for instance, nucleic acid type reverse transcriptase inhibitory agents such as AZT (azido-thymidine). When treating the AIDS-affected patients with these drugs, however, they would produce strong side effects. For instance, it has been known that they inhibit the hemopoietic function or hemocytogenesis of the patients and that they cause anemia in most of the patients.

Under the foregoing circumstances, the present invention has tried to solve the foregoing problems while making the most use of naturally occurring resources.

Patent Documents 1 and 2 specified below disclose that the extracts of plants belonging to the genus Alnus of the family Betulaceae show an anti-aging effect on the skin, but it has not yet been known that these extracts show an influenza virus inhibitory effect, an RS virus inhibitory effect and an immunodeficiency virus inhibitory effect.
Patent Document 1: Japanese Patent No. 3,615,001, Official gazette
Patent Document 2: Japanese Patent No. 2,988,803, Official gazette

### DISCLOSURE OF THE INVENTION

It is an object of the present invention to provide a useful anti-influenza virus agent which comprises, as an effective component, a compound isolated from a plant's element, which shows a strong influenza virus inhibitory effect and which is relatively cheap. It is another object of the present invention to provide a useful anti-RS virus agent which comprises, as an effective component, a compound isolated from a plant's element, which shows a strong RS virus inhibitory effect and which is relatively cheap. Further, it is a still another object of the present invention to provide a useful anti-immunodeficiency virus agent which comprises, as an effective component, a compound isolated from a plant's element, which shows a strong immunodeficiency virus inhibitory effect and which is relatively cheap.

The present invention has been completed on the basis of such a finding that the foregoing problems can be solved by the use of two kinds of plant's components obtained by the inventors of this invention in the course of the studies on the activities of various plant's elements, in other words, the extracts derived (obtained) from plants belonging to the families Betulaceae and/or Meliaceae or dry powdery products of these plants.

More specifically, the present invention herein provides an anti-influenza virus agent which comprises an extract of a plant belonging to the family Betulaceae and/or an extract of a plant belonging to the family Meliaceae. Moreover, the present invention herein provides an anti-RS virus agent which comprises an extract of a plant belonging to the family Betulaceae and/or an extract of a plant belonging to the family Meliaceae. Furthermore, the present invention herein provides an anti-immunodeficiency virus agent which comprises an extract of a plant belonging to the family Betulaceae and/or an extract of a plant belonging to the family Meliaceae.

The present invention also provides an anti-influenza virus agent which comprises dry powder of a plant belonging to the family Betulaceae and/or dry powder of a plant belonging to the family Meliaceae.

The present invention likewise provides an anti-RS virus agent which comprises dry powder of a plant belonging to the family Betulaceae and/or dry powder of a plant belonging to the family Meliaceae.

The present invention further provides an anti-immunodeficiency virus agent which comprises dry powder of a plant belonging to the family Betulaceae and/or dry powder of a plant belonging to the family Meliaceae.

### EFFECT OF THE INVENTION

According to the present invention, there can be provided a useful anti-influenza virus agent which shows a strong influenza virus inhibitory effect and which is relatively cheap. According to the present invention, there can further be provided a useful anti-RS virus agent which shows a strong RS virus inhibitory effect and which is relatively cheap. According to the present invention, there can also be provided a useful anti-immunodeficiency virus agent which shows a strong immunodeficiency virus inhibitory effect and which is relatively cheap.

### BRIEF DESCRIPTION OF THE DRAWINGS

FIG. 1 is a graph showing the variations, with the elapse of time, of the HA value observed for a specimen (an aqueous extract of the plant Alnus diluted 10 times) and a control after the inoculation thereof with H1 human influenza virus (A/PR/8/34).

FIG. 1 is a graph showing the variations, with the elapse of time, of the HA value observed for a specimen (an aqueous extract of the plant Alnus diluted 10 times) and a control after the inoculation thereof with H5 avian influenza virus (A/duck/Singapore-Q/F119-3/97).

### BEST MODE FOR CARRYING OUT THE INVENTION

A group of plants used in the present invention as a raw material are those belonging to the family Betulaceae and they are mainly distributed in the temperate zones. Among them, preferably used in the present invention are, in particular, the plants Alnus. In the present invention, it is more preferred to use the leaves and trunks of the plants belonging to the family Betulaceae. In addition, another group of plants used in the present invention as a raw material are those belonging to the family Meliaceae and they are mainly distributed in the tropic zones, subtropical zones and the temperate zones. Among them, preferably used herein are, in particular, the plants Melia azedarach L. In the present invention, it is more preferred to use the leaves and trunks of the plants belonging to the family Meliaceae. When using these materials as effective components of the anti-influenza virus agent, the anti-RS virus agent or the anti-immunodeficiency virus agent, the plants belonging to the family Betulaceae and the family Meliaceae may be used alone or in combination.

When using, in the present invention, the plants belonging to the family Betulaceae and the family Meliaceae as effective components of the anti-influenza virus agent, the anti-RS virus agent or the anti-immunodeficiency virus agent, the plants can be used in the form of, for instance, a dry powdery product or a granular powdery product obtained by drying the plants and then finely pulverizing them to thus give a solid product such as powder or granules; or they can be used as sawdust generated when the trunk of the plants are cut in round slices with a saw; or the plants can be used in the form of an aqueous extract prepared by directly extracting the plants with water. The amount of the water used in the extraction is not restricted to any particular one, but the water is preferably used in an amount ranging from ⅕ to 10 times and particularly preferably about 2 times the volume of the raw material used. In addition, the extraction is preferably carried out while pulverizing the raw material by stirring the extraction system using, for instance, a mixer. One of ordinary skill in the art can arbitrarily select the stirring time period at his discretion, but it may be, for instance, 5 minutes. After the completion of the stirring operation, the pulverized aqueous mixture may be centrifuged to thus recover the supernatant, which can be used as an extract. One of ordinary skill in the art can arbitrarily select the rotational number and the time period for the centrifugation procedure at his discretion, but they may be, for instance, 4,500 rpm and 20 minutes, respectively. The resulting supernatant can be used without any post-treatment, but it may be stored in its frozen state and sterilized and filtered by passing the supernatant through a sterilization-filtration filter prior to its practical use.

When using the plants belonging to the family Betulaceae and the family Meliaceae in the form of dry powder, it is preferred to pulverize the plants using a mixer after drying them. One of ordinary skill in the art can arbitrarily select the temperature and the time period for the drying operation at his discretion, but the drying operation may, for instance, be carried out at 65° C. overnight. The pulverized and dried powder has a particle size on the order of about 0.2 mm to about 2 mm. After the pulverization, the dry powder can be stored in a container such as a glass bottle containing, for instance, silica gel so that the powder can be maintained in its dry condition.

The anti-influenza virus agent according to the present invention shows its influenza virus inhibitory effect on the whole kinds of influenza viruses including human influenza viruses and avian influenza viruses, but it can particularly show a quite excellent effect on influenza viruses selected from the group consisting of A/Spanish influenza viruses (A/PR/8/34:H1N1), A/Hong Kong influenza viruses (A/Moscow/1/100:H3N2), avian influenza viruses (A/duck/Singapore-Q/F119-3/97:H5N3), and B type influenza viruses (B/Yamagata/16/88). Moreover, the anti-influenza virus agent according to the present invention likewise shows a quite excellent effect on influenza viruses selected from the group consisting of swine influenza virus H1N1 subtype, parainfluenza virus 3 type and parainfluenza virus 1 type.

The anti-immunodeficiency virus agent according to the present invention shows its immunodeficiency virus inhibitory effect on the whole kinds of immunodeficiency viruses including human immunodeficiency virus (HIV) and feline immunodeficiency virus (FIV).

The family retrovirus, to which the immunodeficiency viruses belong, are divided into seven genera. The feline immunodeficiency virus (FIV) is classified as one kind of the viruses belonging to Lentivirus like human immunodeficiency virus type 1 (HIV-1), human immunodeficiency virus type 2 (HIV-2), simian immuno-deficiency virus (SIV), and equine infectious viruses. It has been known that FIV is genetically similar to HIV-1 and HIV-2 in the light of the results of the evolutional analysis of the amino acid sequence of the reverse transcriptase of the virus belonging to the family retrovirus. Moreover, these viruses are also similar to one another in the protein-constituting factors such as gag, pol, vit, rev and env which are important for constituting viruses and accordingly, it has been known that FIV can be used as a model virus in the screening of human immunodeficiency virus inhibitory agents (Fields Virology, 2001, Vol. 2, pp. 2095-2102, Lippincott Williams & Wilkins, Ronald C. Desrosiers).

Incidentally, the anti-influenza virus agent, anti-RS virus agent and anti-immunodeficiency virus agent according to the present invention may likewise comprise, in addition to the foregoing effective component, various kinds of substances, as auxiliary agents, such as an excipient, a diluent, a disintegrating agent, a binder, a coating agent, a lubricant, a sliding agent, a lubricating agent, a flavor, a sweetener, and a plasticizer, which are acceptable from the pharmaceutical standpoint or from the viewpoint of poultry and animal industries. Specific examples thereof include magnesium carbonate; titanium dioxide; lactose, mannitol and other saccharides; talc; milk proteins; gelatin; starch; cellulose and derivatives thereof; animal and vegetable oils; polyethylene glycol; and glycerol.

In the ant-influenza virus agent, anti-RS virus agent and anti-immunodeficiency virus agent according to the present invention, one of ordinary skill in the art can arbitrarily determine, at his discretion, the dilution factor of the extract which is derived from the plants belonging to the family Betulaceae and/or the family Meliaceae and used in the agents as an effective component depending on the intended purposes of the use thereof and the conditions of using the same. For instance, it may preferably range from 2 to 20,000 times, more preferably 10 to 5,000 times, further preferably 500 to 2,000 times the volume of the extract to be used. The anti-influenza virus agent, anti-RS virus agent and anti-immunodeficiency virus agent according to the present invention may be administered through the oral route or through the spray thereof, but the present invention is not restricted to these administration routes at all. When these agents are orally administered, one of ordinary skill in the art can determine, at his discretion, the amount of each of these agents while taking into consideration the intended purposes of the use thereof and the conditions of using the same. For instance, it preferably ranges from 0.01 mL to 40 mL per 1 kg of the body weight, more preferably 0.1 mL to 20 mL per 1 kg of the body weight, and further preferably 0.2 mL to 10 mL per 1 kg of the body weight. One of ordinary skill in the art can likewise determine the amount thereof to be sprayed, at his discretion, while taking into consideration the purposes of the use thereof and the conditions of using the same. For instance, it preferably ranges from 0.001 mL/m² to 50 mL/m², more preferably 0.01 mL/m² to 20 mL/m², and further preferably 0.1 mL/m² to 10 mL/m², on the basis of the area of a subject to be sprayed with each agent.

Similarly, when the dry powder derived from the plants belonging to the families Betulaceae and/or the family Meliaceae is used, as an effective component, in the anti-influenza virus agent, anti-RS virus agent or anti-immunodeficiency virus agent according to the present invention, one of ordinary skill in the art can determine, at his discretion, the amount of the dry powder to be used in these agents while taking into consideration the purposes of the use thereof and the conditions of using the same. For instance, if the anti-influenza virus agent, anti-RS virus agent or anti-immunodeficiency virus agent according to the present invention is used as a disinfectant for the cages positioned in a poultry farm, one of ordinary skill in the art can determine the virus titer of the influenza virus, RS virus or immunodeficiency virus within the subject according to the plaque assay technique disclosed in Examples of this patent application, which makes use of infected cell strains or the titration technique and can then determine the amount of the agent to be used on the basis of the titer thus determined. For instance, the amount, as a unit dose, of the foregoing dry powder preferably ranges from 0.01 mg to 500 g, more preferably 0.1 mg to 100 g, further preferably 0.2 mg to 10 g, still further preferably 2.5 mg to 1 g and most preferably 5 mg to 500 mg per each specific subject.

The anti-influenza virus agent, anti-RS virus agent or anti-immuno-deficiency virus agent according to the present invention can be used, for instance, as an agent for disinfecting the ground and/or the cages in a poultry farm, or the agent may be incorporated into, for instance, a feed for chickens so that the agent may be orally administered to the same. The plants belonging to the families Betulaceae and the family Meliaceae used as an effective component of the anti-influenza virus agent, anti-RS virus agent or anti-immunodeficiency virus agent according to the present invention are highly safe even for the animals and therefore, they are particularly useful. Moreover, the anti-influenza virus agent, anti-RS virus agent or anti-immunodeficiency virus agent according to the present invention can be used as a gargle or an intranasally administrable prophylactic liquid for the prevention of influenza virus-related infectious diseases, RS virus-related infectious diseases or immunodeficiency virus-related infectious diseases, or an agent for disinfecting, for instance, the interiors of each domestic houses, various schools, hospitals and transport facilities, as well as an agent for disinfecting apparatuses and tools for cooking and foods such as meat. When using the anti-influenza virus agent, anti-RS virus agent or anti-immunodeficiency virus agent according to the present invention as a disinfectant, the extracted liquid can be sprayed on a subject to be disinfected through the use of, for instance, a gas-charged spraying can or other sprayers or atomizers. In addition, there can be provided a filtering device effective for inactivating or killing influenza viruses, RS viruses or immunodeficiency viruses if the anti-influenza virus agent, anti-RS virus agent or anti-immunodeficiency virus agent according to the present invention is adsorbed on, for instance, a piece of cloth or a filter used in an aspiration-filtering device. Furthermore, it has also been recognized that the trunks of the plant Alnus and the plant Melia azedarach L. likewise have high influenza virus inhibitory activities and accordingly, when forming cloth or a bag using the fibers of these trunks and packaging dry leaf powder derived from the plants Alnus and/or Melia azedarach L. therein, the resulting goods can be used for removing and sterilizing the air while using them as filters. Moreover, the leaves and trunks of the plants Alnus and/or Melia azedarach L. as well as the dry powder thereof can be used after they are processed using polihexanide hydrochloride and/or surfactants. In this connection, the virus-inactivation effect of the agent would not be deteriorated, but may rather be enhanced by the processing.

The present invention will now be described in more detail with reference to the following Examples.

### EXAMPLE

### Example 1

### Preparation of Sample

### (Extraction of Plant Sample)

After the collection of the leaves of the plant Alnus, the collected green leaves were weighed and then lightly washed with tap water. Pure water was added to the green leaves in an amount of 2 times the volume of the latter and the leaves were pulverized for 5 minutes using a domestic mixer. This liquid pulverized product was centrifuged at 4,500 rpm for 20 minutes to thus recover the resulting supernatant. The supernatant was stored in its frozen state. It was filtered through a sterilization-filtration filter having a pore size of 0.2 μm and then the resulting filtrate of the supernatant was used as a sample in the following tests.

After the collection of the leaves of the plant Melia azedarach L., the collected green leaves were weighed and then washed with running water. The leaves thus washed with water were dried at 65° C. overnight and stored under air tightly sealed and light-shielded conditions till they were practically used. The dried leaves were pulverized in a mill mixer, pure water was added to the dried powder in a rate of 9 mL per 1 g of the latter and then treated in an autoclave under the conditions of 115° C., 30 minutes and 1 kg/cm². Thereafter, the aqueous mixture was treated in an ultrasonic homogenizer for 5 minutes, then subjected to a centrifugation treatment at 5,000 rpm for 30 minutes and the resulting supernatant was recovered. The supernatant was stored in its frozen state till it was practically used. It was filtered through a sterilization-filtration filter having a pore size of 0.2 μm and then the resulting filtrate of the supernatant was used as a sample in the following tests.

### (Drying Treatment of Plant Sample)

After the collection of the leaves of the plant Alnus and those of the plant Melia azedarach L., the respective leaves thus collected were then lightly washed with tap water and they were dried at 65° C. overnight. The dried leaves were finely pulverized using a domestic mill mixer and the finely pulverized leaves were stored in a glass bottle containing silica gel till they were used in the subsequent tests. In addition, after collecting the respective trunks of the plant Alnus and the plant Melia azedarach L., they were cut in round slices with a saw and the sawdust generated during the stage were collected. The sawdust thus collected was dried at 65° C. overnight and stored in a glass bottle containing silica gel before they were used in the following tests.

### Example 2

### In Vitro Evaluation of Influenza Virus Inhibitory Activity

### (Cells and Cultivation Thereof)

Madine Darby Canine Kidney (MDCK) cells derived from the kidney of a dog were subjected to subculture in a 75 cm² flask using an MEM culture medium supplemented with containing 10% fetal calf serum.

### (Preparation of Target Virus-Containing Liquid):

In the evaluation test for the influenza virus inhibitory activity, there were used A/Spanish influenza viruses (A/PR/8/34:H1N1), A/Hong Kong influenza viruses (A/Moscow/1/100:H3N2), avian influenza viruses (A/duck/Singapore-Q/F119-3/97:H5N3), and B type influenza viruses (B/Yamagata/16/88). The sample virus-containing liquids of A/PR/8/34, A/Moscow/1/100 and B/Yamagata/16/88 were prepared as follows. In a 75 cm² flask, MDCK cells were cultivated in an MEM culture medium supplemented with 10% fetal calf serum for 2 days. Then, the cultured cells were washed with PBS, each virus-containing liquid, which had been obtained by the proliferation of the virus using embryonated egg, was diluted 1,000 times with an MEM culture medium containing 5 μg/mL of acetyl trypsin and the viruses thus proliferated were adsorbed on the MDCK cells at 37° C. for 30 minutes. Then, 10 mL of an MEM culture medium containing 5 μg/mL of acetyl trypsin was added and the resulting mixture was allowed to stand at 37° C. for 4 days. After confirming if most of the MDCK cells were infected with the viruses and they were released from the cultivation plane, the culture medium was recovered. The culture medium thus recovered was centrifuged to thus precipitate the residues of the cell, the resulting supernatant was recovered, stored after freezing the same at a temperature of −80° C. and the supernatant thus obtained was used as the virus-containing liquid in the test for inspecting the influenza virus inhibitory activity. A/duck/Singapore-Q/F119-3/97 viruses were proliferated in 11-day-old embryonated egg, the viruses recovered therefrom were stored after freezing the same at −80° C. and used as a virus-containing liquid in the test for inspecting the influenza virus inhibitory activity. Prior to the evaluation test, the foregoing viruses were inspected for the virus titers according to the plaque technique.

### Example 3

### Influenza Virus Inhibitory Activity-Determining Test-1

The in vitro proliferation inhibitory effect of the extract derived from the leaves of the plant Alnus on influenza viruses was evaluated using, as target viruses, A/PR/8/34, A/Moscow/1/100, A/duck/Singapore-Q/F119-3/97 and B/Yamagata/16/88, according to the plaque technique. The MDCK cells used in the evaluation were cultivated, at 37° C. for 2 days, on an MEM culture medium supplemented with 10% fetal calf serum contained in a plastic Petri dish having a diameter of 60 mm. There were blended equivalent amounts of a specimen diluted according to the two-fold serial dilution technique and a virus inoculum diluted to 300 PFU/0.2 mL and the resulting mixture was allowed to stand for 30 minutes. As a control used herein, MEM in an amount identical to that of the specimen was added to the diluted virus inoculum. There were used 2 plastic Petri dishes for each dilution factor. The culture medium was removed from the cultivated cells, the cultivation plane was washed with PBS, the mixed liquid of the specimen and the viruses was added to the cells to thus adsorb the viruses onto the cells for 30 minutes. Thereafter, the inoculum was removed, 5 mL each of an agar culture medium for overlaying was added and the mixture was solidified at room temperature. Thereafter, the cultivation was continued at 37° C. for 3 days, the cells were fixed using a 3.6% formalin, and then stained with Methylene Blue to thus calculate the number of plaques thus formed. The plaque formation-inhibitory rate was calculated on the basis of the number of plaques formed in the specimen-free control test group to thus evaluate each specimen for the influenza virus inhibitory activity. The plaque formation-inhibitory rate was calculated according to the following equation:

Plaque Formation-Inhibitory Rate=[(average plaque number observed at each dilution factor)/(average plaque number observed for control)]×100

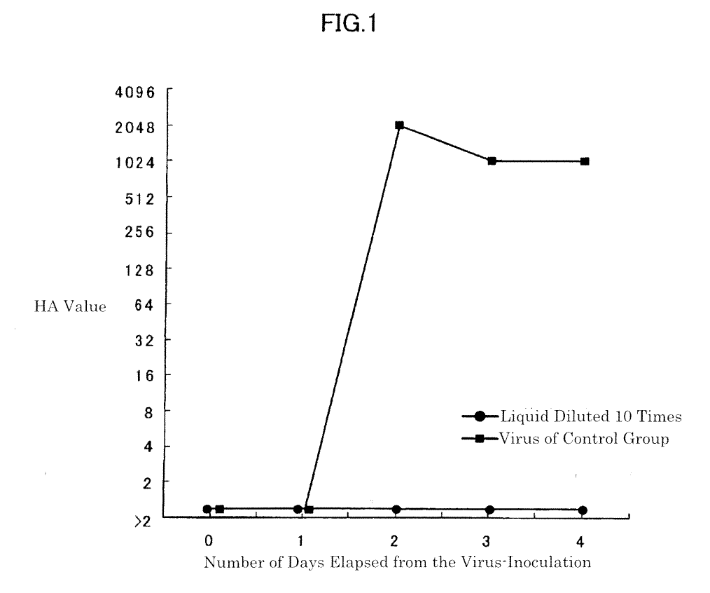

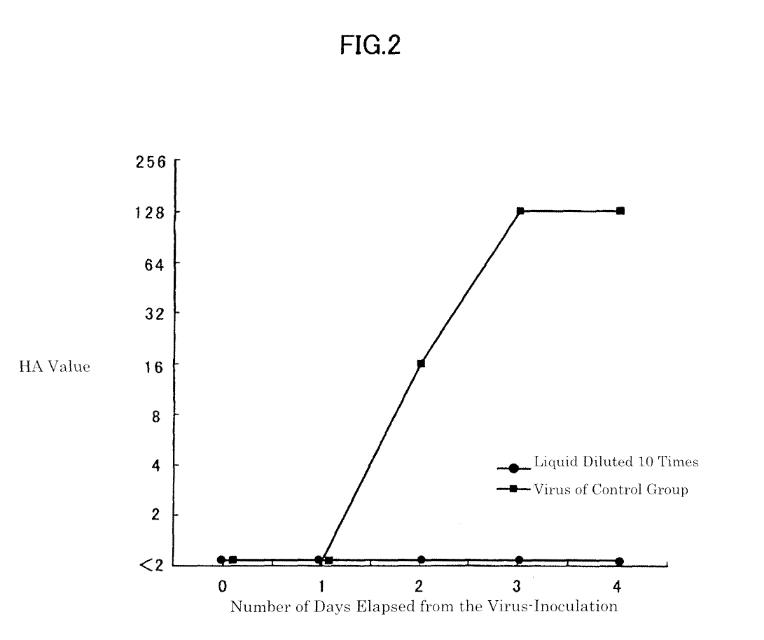

The results thus obtained are plotted on FIG. 1. As will be seen from FIG. 1, the extract derived from the leaves of the plant Alnus can strongly inhibit the plaque formation of wide variety of influenza viruses. The infectious diseases caused by the A/Spanish type strains of influenza viruses prevailed all over the world in human beings during the term extending from 1930's to 1940's. The A/PR/8/34 (H1N1: hereunder referred to as “PR-8”) viruses typical of the foregoing A/Spanish type strains have wisely been used as a typical A type influenza virus which rapidly proliferates within the embryonated egg and the MOCK cells, but the extract of the leaves of the plant Alnus showed a high plaque formation-inhibitory rate, for the foregoing viruses, on the order of not less than 50% even at a quite high dilution factor of not less than 2,000 times. The extract also showed a high plaque formation-inhibitory activity even against the A/Moscow/1/100 as a kind of the A/Hong Kong influenza viruses which have still been prevailing within the terrestrial world. Furthermore, the extract also strongly inhibited the plaque formation of A/duck/Singapore-Q/F119-3/97 (H5N3: H5 avian influenza viruses) belonging to the same strain as the avian H5N1 viruses with which avian has now been infected in various parts of the world and for which it has been confirmed that the human beings have likewise temporarily been infected therewith. In this respect, the plaque formation-inhibitory rate thereof was found to be 1,024 to 2,048. Moreover, the leaves of the plant Melia azedarach L. also shows a plaque formation-inhibitory activity even against B/Yamagata/16/88 as a kind of B type influenza viruses and the plaque formation-inhibitory activity thereof was found to be 512. As has been described above, the extract derived from the leaves of the plant Alnus shows a wide proliferation-inhibitory activity against A and B type influenza viruses and accordingly, the extract was proved to be useful as an effective component of an anti-influenza virus agent.

### Example 4

### Influenza Virus Inhibitory Activity-Determining Test-2

The extracts of the leaves of the plant Alnus and those of the plant Melia azedarach L. were inspected for the in vitro proliferation-inhibitory effect on influenza viruses, with the elapse of time period, using A/PR/8/34 and A/duck/Singapore-Q/F119-3/97 as the target viruses. There were blended equivalent amounts of a specimen diluted 10 and 50 times and a virus-containing liquid adjusted to 300 PFU/0.2 mL and the resulting mixture was allowed to stand at room temperature for 30 minutes. The resulting liquid thus treated was used as a virus inoculum. To determine the proliferation-inhibitory effect, MDCK cells were cultivated, at 37° C. for 2 days, in an MEM culture medium supplemented with 10% fetal calf serum in a plastic Petri dish having a diameter of 100 mm. The culture liquid was removed from the cultivated cells, the cultivation plane was washed with PBS, then 0.2 mL of the virus inoculum was added to the cells to thus adsorb the viruses onto the cells for 30 minutes. Then 10 mL of an MEM culture medium containing 2 μg/mL of trypsin was added thereto. The day on which the cells were inoculated with the virus was set at the 0^{th} day, the viral concentration of the culture medium was evaluated everyday till the 4^{th} day by the determination of the HA value. In the determination of the HA value, a 96-well plastic plate was used, 100 μL each of the specimen was dispensed in the wells of the 1^{st} row of the 96-well plastic plate, while 50 μL each of PBS was likewise dispensed in the wells of the 2^{nd} to 12^{th} rows of the plate. Then 50 μL of the specimen was taken from each of the wells of the 1^{st} row and added to each corresponding well of the 2^{nd} row and these procedures were repeated till the procedures reached the wells of the 12^{th} row to thus stepwise dilute the specimen according to the two-fold serial dilution technique. Then 50 μL each of a 0.5% solution of red blood cells collected from chickens was added to each well, the plate was allowed to stand for 30 minutes and each corresponding HA value was then determined on the basis of the red blood cell-agglutination reaction.

The use of the foregoing experimental system would permit the examination of the anti-viral effect of the extract of the leaves of the plant Alnus, over 4 days, in the cell system in which the proliferation of the virus is continuously advanced. The results thus obtained are plotted on FIGS. 1 and 2. As a result, it became clear that when the experimental system was free of any extract derived from the leaves of the plant Alnus, the A/PR/8/34 virus underwent abrupt proliferation after the 2 days from the inoculation of the virus, the level thereof arrived at a peak value of 2.048 as expressed in terms of the HA activity and a high level on the order of 1.024 was maintained subsequent thereto over 4 days. Similarly, in case of the H5 avian influenza virus (A/duck/Singapore-Q/F119-3/97), the HA value observed on the 2^{nd} day was found to be 16 and the HA value arrived at the peak level of 128 on the 3^{rd} day. On the other hand, when the extract of the leaves of the plant Alnus diluted 10 times was added to the both virus-proliferation systems, the proliferation of these viruses were completely inhibited and any HA activity was not detected at all even on the 4^{th} day. In other words, it was proved that the extract of the leaves of the plant Alnus not only strongly inhibits the plaque formation of the viruses, which serves as an indication of the inhibition of viral proliferation, but also can completely destroy the entire viral proliferation cycle of the human PR88 and avian influenza viruses. The foregoing clearly indicates that the extract of the leaves of the plant Alnus has an effect of inhibiting the infection with the influenza viruses as well as the proliferation thereof.

### Example 5

### Influenza Virus Inhibitory Activity-Determining Test-3

In this test, dry leaves and powder (sawdust) of the plant Alnus and those of the plant Melia azedarach L. (1 g each) were inspected for the abilities to inactivate viruses using A/PR/8/34 and A/duck/Singapore-Q/F119-3/97 as target viruses. First of all, a specimen (1 g each) was introduced into 15 mL volume centrifuge tubes and then virus-containing liquids adjusted to 10², 10³, 10⁴ and 10⁵ PFU/mL respectively were added to these centrifuge tubes. After allowing these tubes to stand at room temperature for 30 minutes, they were centrifuged at 3,000 rpm for 15 minutes to thus recover the corresponding supernatants, each resulting supernatant was filtered through a filtration-sterilization filter having a pore size of 0.2 μm and the resulting filtrates were used as a virus inocula for the determination of plaque infectivity titers. To determine the plaque infectivity titer, MDCK cells were cultivated, at 37° C. for 2 days, in an MEM culture medium supplemented with 10% fetal calf serum in a plastic Petri dish having a diameter of 60 mm. The culture medium was removed from the cultivated cells, the cultivation plane was washed with PBS, 0.2 mL of the virus inoculum prepared above was added to the cells and the viruses were adsorbed on the cells for 30 minutes. Thereafter, the inoculum was removed, 5 mL each of an agar culture medium for overlaying was added and the resulting mixture was solidified at room temperature. Thereafter, the cultivation was continued at 37° C. for 3 days, the cells were fixed using a 3.6% formalin, and then stained with Methylene Blue to thus calculate the number of plaques thus formed. The amount of deactivated viruses was calculated on the basis of the amount of viruses observed for the control test group which had not been treated with any specimen and that observed for the specimen-treated test group according to the following equation:

Amount of Deactivated Viruses (PFU/mL)=[Amount of Viruses observed for Control Group (PFU/mL)]−[Amount of Viruses observed for Specimen-Treated Group (PFU/mL)]

The results thus obtained are plotted on FIG. 2. The data plotted on FIG. 2 clearly indicate that the dry powder of the plant Alnus and that of the plant Melia azedarach L. (1 g each) destroys the infectivity of 28 to 3.6×10⁴ viruses at a rate of 100% and that this effect was recognized over a wide range including human H1 viruses and H5 avian influenza viruses. Accordingly, the use of the and-influenza virus agent according to the present invention would be able to kill avian influenza viruses included in not only the poultry farms, but also the droppings of chickens or the like scattered on the ground surrounding the lakes and marshes in the proximity to the poultry farms to thus prevent the chickens from being infected with the viruses.

In addition, it was also found that the powder (sawdust) of the trunks of the plant Alnus and that of the plant Melia azedarach L. (1 g each) could extinguish the infectivity of influenza viruses on the order of not less than 5.1×10⁴ H1 and that of avian influenza viruses on the order of not less than 3.6×10⁴ H5. Therefore, these powdery products can likewise be used as disinfectants for avian influenza viruses.

### Example 6

### Influenza Virus Inhibitory Activity-Determining Test-4

Dry leaves of the plant Alnus and those of the plant Melia azedarach L. were inspected for the abilities to inactivate viruses using A/PR/8/34 and A/duck/Singapore-Q/F119-3/97 as target viruses, while variously changing the mixing ratio of the dry leaves to a virus-containing liquid whose viral concentration had been adjusted to a predetermined level. The mixing ratio, i.e., specimen: virus-containing liquid was set at 1:20, 1:50, 1:100, 1:200 and 1:1,000. To centrifuge tubes each having a volume of 15 mL, there were added 0.25, 0.1, 0.05, 0.025 and 0.005 g of the specimen and then the virus-containing liquid (adjusted to 10⁶ PFU/mL) was added to each corresponding centrifuge tube in an amount of 4.75, 4.9, 4.95, 4.975 or 4.995 mL. After allowing these centrifuge tubes to stand for 30 minutes, they were centrifuged at 3,000 rpm for 15 minutes and then each resulting supernatant was recovered. Each of these supernatants was filtered through a filtration-sterilization filter having a pore size of 0.2 μm and the resulting filtrate was used as a virus inoculum. The same procedures used in the foregoing Test-1 were repeated to determine the plaque infectivity titer to thus calculate each corresponding amount of deactivated viruses.

The results thus obtained are plotted on FIG. 3. As a result, it was proved that the dry leaves of the plant Alnus can extinguish such an extremely high virus infectivity titer of 5.02×10⁵ while using the specimen in such a small amount of 0.005 g.

In addition, it was also proved that the dry leaves can extinguish very high virus infectivity titer on the order of 5.05×10⁵ while using the specimen in an amount of 0.0025 g. Furthermore, when using the specimen in an amount of 0.05 g, it could completely kill not less than 5.05×10⁵ viruses. On the other hand, when using the dry leaves of the plant Melia azedarach L., it was proved that the specimen thereof can completely kill not less than 5.05×10⁵ viruses in a rate of 100% while using the specimen in an amount of 0.005 g and that the dry leaves of the plant Melia azedarach L. has a virus-deactivation ability higher than that observed for the dry leaves of the plant Alnus.

TABLE 1
Proliferation-Inhibitory Effect of Extract Derived from
leaves of Plant Alnus on Influenza Viruses

| | ≧50% Plaque |
|---|---|
| | Formation- Inhibitory |
| Virus | Activity^{#} |
| A/Spanish Type: A/PR/8/34 (H1N1) | 2,048-4,096 |
| A/Hong Kong Type: A/Moscow/1/100 (H3N2) | 1.024-2,048 |
| Avian influenza: A/duck/Singapore-Q/F119- | 1.024-2,048 |
| 3/97 (H5N3) | |
| B Type: B/Yamagata/16/88 | 512 |

^{#}The plaque formation-inhibitory activity is expressed in terms of the maximum dilution factor of each specimen showing a plaque formation-inhibitory activity of not less than 50%.

TABLE 2
Abilities of Dry Leaves and Powdered Trunks of Alnus and Melia
azedarach L. to Deactivate Avian and Human Influenza Viruses

| | | Infectivity Titer of Viruses Tested | | | |
|---|---|---|---|---|---|
| | | (PFU/mL) | | | |
| | | | Amt. | Resid- | Amt. of |
| Specimen | | | Chal- | ual | Viruses |
| Kind | Amt. | Kind | lenged | Amt. | Deactivated |
| Leaves of | 1 g | H1 Human | 5.1 × 10⁴ | 0 | >5.1 × 10⁴ |
| the Plant | 1 g | Influenza Virus^{#} | 3.9 × 10³ | 0 | >3.9 × 10³ |
| Alnus | 1 g | | 1.7 × 10² | 0 | >1.7 × 10² |
| | 1 g | | 1.3 × 10 | 0 | >1.3 × 10 |
| | 1 g | H5 Avian | 3.6 × 10⁴ | 0 | >3.6 × 10⁴ |
| | 1 g | Influenza Virus* | 3.6 × 10³ | 0 | >3.6 × 10³ |
| | 1 g | | 3.8 × 10² | 0 | >3.8 × 10² |
| | 1 g | | 2.8 × 10 | 0 | >2.8 × 10 |
| Leaves of | 1 g | H1 Human | 2.5 × 10⁴ | 0 | >2.5 × 10⁴ |
| the Plant | 1 g | Influenza Virus | 2.0 × 10³ | 0 | >2.0 × 10³ |
| Melia | 1 g | | 1.7 × 10² | 0 | >1.7 × 10² |
| azedarach | 1 g | | 1.3 × 10 | 0 | >1.3 × 10 |
| L. | 1 g | H5 Avian | 1.7 × 10⁵ | 0 | >1.7 × 10⁵ |
| | 1 g | Influenza Virus | 1.5 × 10⁴ | 0 | >1.5 × 10⁴ |
| | 1 g | | 1.7 × 10³ | 0 | >1.7 × 10³ |
| | 1 g | | 1.1 × 10² | 0 | >1.1 × 10² |
| Trunks of | 1 g | H1 Human | 5.1 × 10⁴ | 0 | >5.1 × 10⁴ |
| Alnus | | Influenza Virus | | | |
| | 1 g | H5 Avian | 3.6 × 10⁴ | 0 | >3.6 × 10⁴ |
| | | Influenza Virus | | | |
| Trunks of | 1 g | H1 Human | 5.1 × 10⁴ | 0 | >5.1 × 10⁴ |
| Melia | | Influenza Virus | | | |
| azedarach | 1 g | H5 Avian | 3.6 × 10⁴ | 0 | >3.6 × 10⁴ |
| L. | | Influenza Virus | | | |

^{#}The human influenza virus used herein was A/PR/8/34 (H1N1) strain;
*The avian influenza virus used herein was A/duck/Singapore-Q/F119-3/97 (H5N3) strain.

TABLE 3
Amounts of H1 Influenza Viruses Deactivated by Dry
Powder of Alnus and Melia azedarach L.

| | | Infectivity Titer of Viruses Tested^{#} | | |
|---|---|---|---|---|
| | | (PFU/mL) | | |
| Specimen | | Amount | Resid- | Amt. of |
| | Amount | Chal- | ual | Virus |
| Kind | (g) | lenged | Amt. | Deactivated |
| Dry Powder | 0.250 | 5.05 × 10⁵ | 0 | >5.05 × 10⁵ |
| Derived from | 0.100 | 5.05 × 10⁵ | 0 | >5.05 × 10⁵ |
| Alnus | 0.050 | 5.05 × 10⁵ | 0 | >5.05 × 10⁵ |
| | 0.025 | 5.05 × 10⁵ | 0 | 5.05 × 10⁵ |
| | 0.005 | 5.05 × 10⁵ | 2.8 × 10³ | 5.02 × 10⁵ |
| Dry Powder | 0.250 | 5.4 × 10⁵ | 0 | >5.4 × 10⁵ |
| Derived from | 0.100 | 5.4 × 10⁵ | 0 | >5.4 × 10⁵ |
| Melia azedarach | 0.050 | 5.4 × 10⁵ | 0 | >5.4 × 10⁵ |
| L. | 0.025 | 5.05 × 10⁵ | 0 | >5.05 × 10⁵ |
| | 0.005 | 5.05 × 10⁵ | 0 | >5.05 × 10⁵ |

^{#}The influenza virus used herein was A/PR/8/34 (H1N1) strain.

### Example 7

### Influenza Virus Inhibitory Activity-Determining Test-5 (Swine Influenza Virus H1N1 Subtype)

A/Swine/88 strain of swine influenza virus H1N1 subtype was diluted 100 times, inoculated into MDCK cells, and blended with an equivalent volume of the extract of the plant Melia azedarach L. (product prepared in Example 1), which had stepwise been diluted according to the two-fold serial dilution technique 2 prior to the inoculation. After the infection with the virus, the blend was inspected for the viral proliferation, over 5 days, using the HA activity as an indication. As a result, the number of the viruses in the control group which was free of any treatment with the extract of Melia azedarach L. reached 16 times after 2 days, 64 times after 3 days and further 64 times after 4 days. Contrary to this, the proliferation of the virus was not detected at all in the test group treated with the extract of Melia azedarach L. during the testing period over 5 days and this clearly indicates that the extract of Melia azedarach L. completely inhibits the proliferation of the swine influenza virus.

### Example 8

### Influenza Virus Inhibitory Activity-Determining Test-6 (Parainfluenza Virus 3 Type and Parainfluenza Virus 1 Type)

The extract of the plant Alnus and that of the plant Melia azedarach L. (products prepared in Example 1) were inspected for the effect of inhibiting the proliferation of parainfluenza virus 3 type and parainfluenza virus 1 type according to the following method: Parainfluenza virus 3 type (Toshiba strain) belonging to the family Paramyxoviridae was manipulated to form virus inocula each having a virus density ranging from 200 to 400 plaque-forming units, the extract of the plant Melia azedarach L. which had been diluted according to the two-fold serial dilution was added to the virus inoculum in an amount equivalent to the latter, followed by the reaction therebetween for 30 minutes, the inoculation of the resulting product into VERO cells, the cultivation of the cells over 3 days, the determination of the number of plaques thus formed and the calculation of the rate of plaque formation inhibition on the basis of the resulting plaque analysis. The results thus obtained are summarized in the following Table 4. As a result, it was recognized that, regarding the plaque formation inhibitory rate of the extract derived from the plant Melia azedarach L., the extract shows a high virus-proliferation inhibitory rate of not less than 50% higher, even when the extract is diluted 32,768 times and this clearly indicates that the components of the plant Melia azedarach L. can quite efficiently inhibit the proliferation of the parainfluenza virus 3 type.

Moreover, it was also proved that the extract of the plant Melia azedarach L. also shows an effect of inhibiting the proliferation of parainfluenza virus 1 type. Furthermore, the same procedures used above were repeated to inspect the extract of the plant Alnus (the product prepared in Example 1) for the effect of inhibiting the proliferation of parainfluenza viruses and as a result, the components of the extract were found to show an extremely high proliferation inhibitory effect against parainfluenza virus 3 type (Name of strain: 57-34) such that the plaque formation inhibitory rate was not less than 50% at a dilution factor of 512. From the foregoing, the proliferation-inhibitory effect of the extract of the plant Alnus was found to be about 1/100 time that observed for the extract of the plant Melia azedarach L., but the foregoing fact clearly indicates that the extract of Alnus distinctly inhibits the proliferation of parainfluenza viruses. This strongly suggests that the components of the plant Melia azedarach L. and the plant Alnus would influence the proliferation of most of the viruses belonging to the family Paramyxoviridae and it would be believed that they can inhibit the proliferation of, in particular, RS viruses belonging to the family. These results strongly suggest that the extract according to the present invention is quite useful as a disinfectant for extinguishing or killing parainfluenza viruses and RS viruses which may arise a serious problem of the hospital infection.

TABLE 4
Plaque Formation-inhibitory Effect of Extracts of Alnus and
Melia azedarach L. against Parainfluenza Virus 3 Type:

| Kind of | | Sample-Dilution Factor | | | | | | | |
|---|---|---|---|---|---|---|---|---|---|
| Plant | Plaque Formation | 8 | 32 | 128 | 512 | 2,048 | 8,192 | 32,768 | 131,072 |
| Melia | Number of Plaques/mL | 0 | 0 | 40 | 47.5 | 237.5 | 222.5 | 345 | 497.5 |
| azedarach L. | Plaque Formation-Inhibitory Rate (%) | 100 | 100 | 95.5 | 94.6 | 73.1 | 74.8 | 60.9 | 43.6 |
| Alnus | Number of Plaques/mL | 7.5 | 15 | 60 | 230 | 452.5 | 485 | 642.5 | ND |
| | Plaque Formation-Inhibitory Rate (%) | 99.2 | 98.3 | 93.2 | 73.9 | 48.7 | 45 | 27.2 | ND |

ND: Not tested.

### Example 9

### Immunodeficiency Virus Inhibitory Activity-Determining Test (Proliferation-Inhibitory Effect of Extract of Melia azedarach L. against Immunodeficiency Virus)

To inspect the components of the plant Melia azedarach L. for the effect of inhibiting the proliferation of immunodeficiency virus of the family Retroviridae, tests were carried out according to the following method, while using, as a substitute for human immunodeficiency virus, feline immunodeficiency virus FIV. In this respect, the feline immunodeficiency virus FIV has been known that it can be used as a model virus in the screening of a human immunodeficiency virus inhibitory agent. The extract of the plant Melia azedarach L. was added to feline immunodeficiency virus which had been diluted 100 times, they were reacted with each other for 30 minutes, the reaction system was inoculated into CrFK cells (Crandell Feline Kidney (CrFK) cells), and the cells were inspected for the appearance or development of the cytopathic effect (CPE) generated due to the infection with the virus. The results obtained are listed in the following Table 5. The results indicate that the cytopathic effect generated due to the infection with the feline immunodeficiency virus is completely inhibited even when using the sample diluted 64 times. The foregoing results would strongly suggest that the extract of the plant Melia azedarach L. shows significant killing effect against wide variety of envelope viruses including immunodeficiency viruses and that the extract can be used as useful disinfectants and therapeutic-preventive agents.

TABLE 5
Proliferation-Inhibitory Effect of Extract of
Melia azedarach L. against Feline AIDS Viruses

| | Sample Dilution Factor | | | | |
|---|---|---|---|---|---|
| | 8 | 16 | 32 | 64 | 128 |
| Rate of CPE Development | 0/5 | 0/5 | 0/5 | 0/5 | 5/5 |

* A mixed liquid of the virus and the sample was inoculated to the cell culture in a rate of 5 wells for each dilution factor. Each numerator appearing in Table 5 is the numerical value representing the cytopathic effect, while the denominator represents the number of cell cultures used in each test.

1. An anti-influenza virus agent comprising an extract derived from a plant belonging to the family Betulaceae and/or a plant belonging to the family Meliaceae.

2. The anti-influenza virus agent as set forth in claim 1, wherein the extract of a plant belonging to the family Betulaceae is an aqueous extract derived from the leaves or trunks of the plant Alnus.

3. The anti-influenza virus agent as set forth in claim 1, wherein the extract of a plant belonging to the family Meliaceae is an aqueous extract derived from the leaves or trunks of the plant Melia azedarach L.

4. An anti-influenza virus agent comprising dry powder of a plant belonging to the family Betulaceae and/or a plant belonging to the family Meliaceae.

5. The anti-influenza virus agent as set forth in claim 4, wherein the dry powder of a plant belonging to the family Betulaceae is dry powder of the leaves or trunks of the plant Alnus.

6. The anti-influenza virus agent as set forth in claim 4, wherein the dry powder of a plant belonging to the family Meliaceae is dry powder of the leaves or trunks of the plant Melia azedarach L.

7. The anti-influenza virus agent as set forth in claim 1, wherein the influenza virus is a member selected from the group consisting of A/Spanish influenza viruses (A/PR/8/34:H1N1), A/Hong Kong influenza viruses (A/Moscow/1/100:H3N2), avian influenza viruses (A/duck/Singapore-Q/F119-3/97:H5N3), and B type influenza viruses (B/Yamagata/16/88).

8. The anti-influenza virus agent as set forth in claim 1, wherein the influenza virus is a member selected from the group consisting of swine influenza virus H1N1 subtype, parainfluenza virus 3 type and parainfluenza virus 1 type.

9. An anti-RS virus agent comprising an extract derived from a plant belonging to the family Betulaceae and/or a plant belonging to the family Meliaceae.

10. An anti-RS virus agent comprising dry powder of a plant belonging to the family Betulaceae and/or a plant belonging to the family Meliaceae.

11. An anti-immunodeficiency virus agent comprising an extract derived from a plant belonging to the family Betulaceae and/or a plant belonging to the family Meliaceae.

12. An anti-immunodeficiency virus agent comprising dry powder of a plant belonging to the family Betulaceae and/or a plant belonging to the family Meliaceae.
